(19) United States
(12) Patent Application Publication
(10) Pub. No.: US 2009/0221566 A1
(43) Pub. Date: Sep. 3, 2009

(54) INHIBITORS OF PHOSPHODIESTERASE TYPE-IV

(76) Inventors: Venkata P. Palle, Pune (IN); Sarala Balachandran, Mumbia (IN); Sarika Ramnani, Lucknow (IN); Abhijit Ray, New Delhi (IN); Sunanda G. Dastidar, New Delhi (IN)

Correspondence Address: RANBAXY INC.;INTELLECTUAL PROPERTY DEPT., 600 COLLEGE ROAD EAST, SUITE 2100, PRINCETON, NJ, 08540, US

(21) Appl. No.: 12/090,790

(22) PCT Filed: Oct. 5, 2006
(86) PCT No.: PCT/IB2006/053652

§ 371 Date: Jul. 30, 2008

(30) Foreign Application Priority Data
Oct. 19, 2005 (IN) 2795/DEL/2005

(51) Int. Cl. A61K 31/537; C07D 265/02; A61K 31/535; A61P 25/00; A61P 11/06; A61P 11/08; A61P 17/00; A61P 17/06; A61P 19/02; A61P 19/10

(52) U.S. Cl. 514/230.5; 544/63; 514/228.8; 544/71

(57) ABSTRACT

The present invention relates to oxazine derivatives, which can be used as selective inhibitors of phosphodiesterase (PDE) type IV. Compounds disclosed herein can be useful in the treatment of CMS disorders, AIDS, asthma, arthritis, bronchitis, chronic obstructive pulmonary disease (COPD), psoriasis, allergic rhinitis, shock, atopic dermatitis, Crohn's disease, adult respiratory distress syndrome (ARDS), eosinophilic granuloma, allergic conjunctivitis, osteoarthritis, ulcerative colitis and other inflammatory diseases especially in humans. Processes for the preparation of disclosed compounds are provided, as well as pharmaceutical compositions containing the disclosed compounds, and their use as phosphodiesterase (PDE) type IV inhibitors.

### FIELD OF THE INVENTION

The present invention relates to oxazine derivatives, which can be used as selective inhibitors of phosphodiesterase (PDE) type IV.

Compounds disclosed herein can be useful in the treatment of CNS disorders, AIDS, asthma, arthritis, bronchitis, chronic obstructive pulmonary disease (COPD), psoriasis, allergic rhinitis, shock, atopic dermatitis, Crohn's disease, adult respiratory distress syndrome (ARDS), eosinophilic granuloma, allergic conjunctivitis, osteoarthritis, ulcerative colitis and other inflammatory diseases especially in humans.

Processes for the preparation of disclosed compounds are provided, as well as pharmaceutical compositions containing the disclosed compounds, and their use as phosphodiesterase (PDE) type IV inhibitors.

### BACKGROUND OF THE INVENTION

It is known that cyclic adenosine-3′,5′-monophosphate (cAMP) exhibits an important role of acting as an intracellular secondary messenger (Sutherland, et al. Pharmacol. Rev., (1960), 12, 265). Its intracellular hydrolysis to adenosine 5′-monophosphate (AMP) causes number of inflammatory conditions which are not limited to psoriasis, allergic rhinitis, shock, atopic dermatitis, Crohn's disease, adult respiratory distress syndrome (ARDS), eosinophilic granuloma, allergic conjunctivitis, osteoarthritis, ulcerative colitis. The most important role in the control of cAMP (as well as of cGMP) levels is played by cyclic nucleotide phosphodiesterases (PDE) which represent a biochemically and functionally highly variable superfamily of the enzyme; eleven distinct families with more than 25 gene products are currently recognized. Although PDE I, PDE II, PDE III, PDE IV, and PDE VII all use cAMP as a substrate, only the PDE IV and PDE VII types are highly selective for hydrolysis of c AMP. Inhibitors of PDE, particularly the PDE IV inhibitors, such as rolipram or Ro-1724 are therefore known as cAMP-enhancers. Immune cells contain type IV and type III PDE, the PDE IV type being prevalent, in human mononuclear cells. Thus the inhibition of phosphodiesterase type IV has been a target for modulation and, accordingly, for therapeutic intervention in a range of disease processes.

The initial observation that xanthine derivatives, theophylline and caffeine inhibit the hydrolysis of cAMP led to the discovery of the required hydrolytic activity in the cyclic nucleotide phosphodiesterase (PDE) enzymes. More recently, distinct classes of PDEs have been recognized (Bervo, et al., TIPS, (1990), 11, 150), and their selective inhibition has led to improved drug therapy (Nicholus et al., TIPS, (1991), 12, 19). Thus it was recognized that inhibition of PDE IV could lead to inhibition of inflammatory mediator release (Verghese et. al., J. Mol. Cell. Cardiol., (1989), 12 (Suppl.II), S 61).

WO2005/021515 discloses inhibitors of phosphodiesterase type-IV.

### SUMMARY OF THE INVENTION

The present invention provides oxazine derivatives, which can be used for the treatment of, for example, CNS disorders, AIDS, asthma, arthritis, bronchitis, chronic obstructive pulmonary disease (COPD), psoriasis, allergic rhinitis, shock, atopic dermatitis, Crohn's disease, adult respiratory distress syndrome (ARDS), eosinophilic granuloma, allergic conjunctivitis, osteoarthritis, ulcerative colitis and other inflammatory diseases, and the processes for the synthesis of these compounds.

Pharmaceutically acceptable salts, pharmaceutically acceptable solvates, enantiomers, diastereomers or N-oxides of these compounds having the same type of activity are also provided.

Pharmaceutical compositions containing the compounds, which may also contain pharmaceutically acceptable carriers or diluents, can be used for the treatment of CNS disorders, AIDS, asthma, arthritis, bronchitis, chronic obstructive pulmonary disease (COPD), psoriasis, allergic rhinitis, shock, atopic dermatitis, Crohn's disease, adult respiratory distress syndrome, eosinophilic granuloma, allergic conjunctivitis, osteoarthritis, ulcerative colitis and other inflammatory diseases.

Other aspects will be set forth in the accompanying description which follows and in part will be apparent from the description or may be learnt by the practice of the invention.

In accordance with one aspect, a compound is provided having the structure of Formula Ia

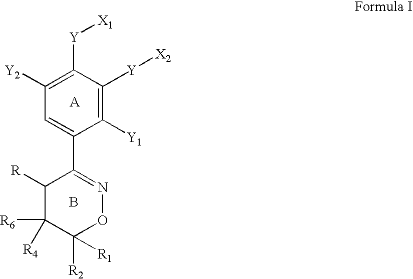

and its pharmaceutically acceptable salts, pharmaceutically acceptable solvates, enantiomers, diastereomers or N-oxides, wherein

R₁ can be hydrogen; alkyl; heterocyclyl; —(CH₂)₁₋₄OR′; —C(═O)NRₓR_{y} or —(CH₂)ₘ—C(═O)R₃;

m is an integer from 0-4;

R₂ can be —(CH₂)ₘC(═O)R₃; —(CH₂)₁₋₄OR′; —C(═O)NRₓR_{y} or R₁ and R₂ together forms an optionally substituted cycloalkyl or heterocyclyl ring system;

R₃ is OR′ or R′;

R₄ can be hydrogen; alkyl; —OR₅; halogen; —NH₂, substituted amino; cyano; carboxy; or —C(═O)NRₓR_{y}; or R₂ and R₄ together joins to form optionally substituted cycloalkyl or heterocyclyl ring system;

R₆ can be hydrogen; alkyl; alkenyl; alkynyl; —OR₅; halogen; cyano; —NH₂ or substituted amino; or R₄ and R₆ together joins to form optionally substituted cycloalkyl or heterocyclyl ring system;

R′ can be alkyl; alkenyl; alkynyl; aryl; cycloalkyl; heteroaryl; heterocyclyl; heteroarylalkyl; heterocyclyl alkyl or aralkyl;

R is R′; —OR₅; halogen; cyano; —NH₂ or substituted amino.

X₁ and X₂ each independently can be hydrogen; alkyl; alkaryl; cycloalkyl; heterocyclyl; heteroaryl; heterocyclylalkyl or heteroarylalkyl;

Y can be an oxygen atom; a sulphur atom; or —NR;

Y₁ and Y₂ each independently can be hydrogen; alkyl; —OR; —SR; or —NHR;

wherein any of Y₁ and X₂ & X₁ and Y₂ together optionally form a cyclic ring fused with the ring A, the ring containing 3-5 carbon atoms within the ring and having 1-3 heteroatoms such as N, O and S;

X₁ and X₂ can alternatively together optionally forms a cyclic ring fused with the ring A, the ring containing 3-5 carbon atoms within the ring and having 2-3 heteroatoms such as N, O and S; and

Rₓ and R_{y} can be hydrogen, alkyl, alkenyl of three to six carbon atoms, alkynyl of three to six carbon atoms, cycloalkyl, —SO₂R₅ (wherein R₅ is hydrogen, alkyl, alkenyl, alkynyl, aryl, cycloalkyl, alkaryl, heteroaryl, heteroarylalkyl, heterocyclyl or heterocyclylalkyl), aryl, alkaryl, heteroaryl, heterocyclyl, heteroarylalkyl, and heterocyclylalkyl.

The following definitions apply to terms as used herein,

The term “alkyl,” unless otherwise specified, refers to a monoradical branched or unbranched saturated hydrocarbon chain having from 1 to 20 carbon atoms. Alkyl groups can be optionally interrupted by atom(s) or group(s) independently selected from oxygen, sulfur, a phenylene, sulphinyl, sulphonyl group or —NR_{α}—, wherein R_{α} can be hydrogen, alkyl, cycloalkyl, alkenyl, cycloalkenyl, alkynyl, aryl, acyl, aralkyl, —C(═O)OR_{λ}, SOₘR_{ψ} or —C(═O)NR_{λ}R_{π}. This term can be exemplified by groups such as methyl, ethyl, n-propyl, iso-propyl, n-butyl, iso-butyl, sec-butyl, t-butyl, n-pentyl, isopentyl, neopentyl, n-hexyl, n-decyl, tetradecyl, and the like. Alkyl groups may be substituted further with one or more substituents selected from alkenyl, alkynyl, alkoxy, cycloalkyl, cycloalkenyl, acyl, acylamino, acyloxy, alkoxycarbonylamino, azido, cyano, halogen, hydroxy, keto, oxo, thiocarbonyl, carboxy, carboxyalkyl, aryl, heterocyclyl, heteroaryl, (heterocyclyl)alkyl, cycloalkoxy, —CH═N—O(C₁₋₆alkyl), —CH═N—NH(C₁₋₆alkyl), —CH═N—NH(C₁₋₆alkyl)-C₁₋₆alkyl, arylthio, thiol, alkylthio, aryloxy, nitro, aminosulfonyl, aminocarbonylamino, —NHC(═O)R_{λ}, —NR_{λ}R_{π}, —C(═O)NR_{λ}R_{π}, —NHC(═O)NR_{λ}R_{π}, —C(═O)heteroaryl, C(═O)heterocyclyl, —O—C(═O)NR_{λ}R_{π} {wherein R_{λ} and R_{π} are independently selected from hydrogen, halogen, hydroxy, alkyl, alkenyl, alkynyl, alkenyl, alkoxy, cycloalkyl, cycloalkenyl, aryl, aralkyl, heterocyclyl, heteroaryl, heterocyclylalkyl, heteroarylalkyl or carboxy}, nitro or —SOₘR_{ψ} (wherein m is an integer from 0-2 and R_{ψ} is hydrogen, alkyl, alkenyl, alkynyl, cycloalkyl, aralkyl, aryl, heterocyclyl, heteroaryl, heteroarylalkyl or heterocyclylalkyl). Unless otherwise constrained by the definition, alkyl substituents may be further substituted by 1-3 substituents selected from alkyl, alkenyl, alkynyl, carboxy, —NR_{λ}R_{π}, —C(═O)NR_{λ}R_{π}, —OC(═O)NR_{λ}R_{π}, —NHC(═O)NR_{λ}R_{π}, hydroxy, alkoxy, halogen, CF₃, cyano, and —SOₘR_{ψ}; or an alkyl group also may be interrupted by 1-5 atoms of groups independently selected from oxygen, sulfur or —NR_{α}— (wherein R_{α}, R_{λ}, R_{π}, m and R_{ψ} are the same as defined earlier). Unless otherwise constrained by the definition, all substituents may be substituted further by 1-3 substituents selected from alkyl, alkenyl, alkynyl, carboxy, carboxyalkyl, —NR_{λ}R_{π}, —C(═O)NR_{λ}R_{π}, —O—C(═O)NR_{λ}R_{π}, hydroxy, alkoxy, halogen, CF₃, cyano, and —SOₘR_{ψ} (wherein R_{λ}, R_{π}, m and R_{ψ} are the same as defined earlier); or an alkyl group as defined above that has both substituents as defined above and is also interrupted by 1-5 atoms or groups as defined above.

The term “alkenyl,” unless otherwise specified, refers to a monoradical of a branched or unbranched unsaturated hydrocarbon group having from 2 to 20 carbon atoms with cis, trans or geminal geometry. Alkenyl groups can be optionally interrupted by atom(s) or group(s) independently chosen from oxygen, sulfur, phenylene, sulphinyl, sulphonyl and —NR_{α}— (wherein R_{α} is the same as defined earlier). In the event that alkenyl is attached to a heteroatom, the double bond cannot be alpha to the heteroatom. Alkenyl groups may be substituted further with one or more substituents selected from alkyl, alkenyl, alkynyl, alkoxy, cycloalkyl, cycloalkenyl, acyl, acylamino, acyloxy, —NHC(═O)R_{λ}, —NR_{λ}R_{π}, —C(═O)NR_{λ}R_{π}, —NHC(O)NR_{λ}R_{π}, —O—C(═O)NR_{λ}R_{π}, alkoxycarbonylamino, azido, cyano, halogen, hydroxy, oxo, keto, carboxyalkyl, thiocarbonyl, carboxy, arylthio, thiol, alkylthio, aryl, aralkyl, aryloxy, heterocyclyl, heteroaryl, heterocyclyl alkyl, heteroaryl alkyl, aminosulfonyl, aminocarbonylamino, alkoxyamino, hydroxyamino, alkoxyamino, nitro or SOₘR_{ψ} (wherein R_{λ}, R_{π}, m and R_{ψ} are as defined earlier). Unless otherwise constrained by the definition, alkenyl substituents optionally may be substituted further by 1-3 substituents selected from alkyl, alkenyl, alkynyl, carboxy, hydroxy, alkoxy, halogen, —CF₃, cyano, —NR_{λ}R_{π}, —C(═O)NR_{λ}R_{π}, —O—C(O)NR_{λ}R_{π} and —SOₘR_{ψ} (wherein R_{λ}, R_{π}, m and R_{ψ} are as defined earlier). Groups, such as ethenyl or vinyl (CH═CH₂), 1-propylene or allyl (—CH₂CH═CH₂), iso-propylene (—C(CH₃)═CH₂), bicyclo[2.2.1]heptene, and the like, exemplify this term.

The term “alkynyl,” unless otherwise specified, refers to a monoradical of an unsaturated hydrocarbon, having from 2 to 20 carbon atoms. Alkynyl groups can be optionally interrupted by atom(s) or group(s) independently chosen from oxygen, sulfur, phenylene, sulphinyl, sulphonyl and —NR_{α}— (wherein R_{α} is the same as defined earlier). In the event that alkynyl groups are attached to a heteroatom, the triple bond cannot be alpha to the heteroatom. Alkynyl groups may be substituted further with one or more substituents selected from alkyl, alkenyl, alkoxy, cycloalkyl, cycloalkenyl, acyl, acylamino, acyloxy, alkoxycarbonylamino, azido, cyano, halogen, hydroxy, keto, oxo, thiocarbonyl, carboxy, carboxyalkyl, arylthio, thiol, alkylthio, aryl, aralkyl, aryloxy, aminosulfonyl, aminocarbonylamino, hydroxyamino, alkoxyamino, nitro, heterocyclyl, heteroaryl, heterocyclylalkyl, heteroarylalkyl, —NHC(═O)R_{λ}, —NR_{λ}R_{π}, —NHC(═O)NR_{λ}R_{π}, —C(═O)NR_{λ}R_{π}, —O—C(═O)NR_{λ}R_{π} or —SOₘR_{ψ} (wherein R_{λ}, R_{π}, m and R_{ψ} are the same as defined earlier). Unless otherwise constrained by the definition, alkynyl substituents optionally may be substituted further by 1-3 substituents selected from alkyl, alkenyl, alkynyl, carboxy, carboxyalkyl, hydroxy, alkoxy, halogen, CF₃, —NR_{λ}R_{π}, —C(═O)NR_{λ}R_{π}, —NHC(═O)NR_{λ}R_{π}, —C(═O)NR_{λ}R_{π}, cyano or —SOₘR_{ψ} (wherein R_{λ}, R_{π}, m and R_{ψ} are the same as defined earlier).

The term “alkoxy” denotes the group Q-alkyl wherein alkyl is the same as defined above.

The term “aryl,” unless otherwise specified, refers to aromatic system having 6 to 14 carbon atoms, wherein the ring system can be mono-, bi- or tricyclic and are carbocyclic aromatic groups. For example, aryl groups include, but are not limited to, phenyl, biphenyl, anthryl or napthyl ring and the like, optionally substituted with 1 to 3 substituents selected from halogen (e.g., F, Cl, Br, I), hydroxy, alkyl, alkenyl, alkynyl, cycloalkyl, alkoxy, acyl, aryloxy, CF₃, cyano, nitro, COOR_{ψ}, NHC(═O)R_{λ}, —NR_{λ}R_{π}, —C(O)NR_{λ}R_{π}, —NHC(═O)NR_{λ}R_{π}, —O—C(═O)NR_{λ}R_{π}, —SOₘR_{ψ}, carboxy, heterocyclyl, heteroaryl, heterocyclylalkyl, heteroarylalkyl or amino carbonyl amino, mercapto, haloalkyl, optionally substituted aryl, optionally substituted heterocyclylalkyl, thioalkyl, —CONHR_{π}, —OCOR_{π}, —COR_{π}, —NHSO₂R_{π} or —SO₂NHR_{π} (wherein R_{λ}, R_{π}, m and R_{ψ} are the same as defined earlier). Aryl groups optionally may be fused with a cycloalkyl group, wherein the cycloalkyl group may optionally contain heteroatoms selected from O, N or S. Groups such as phenyl, naphthyl, anthryl, biphenyl, and the like exemplify this term.

The term “aralkyl,” unless otherwise specified, refers to alkyl-aryl linked through an alkyl portion (wherein alkyl is as defined above) and the alkyl portion contains 1-6 carbon atoms and aryl is as defined below. Examples of aralkyl groups include benzyl, ethylphenyl, propylphenyl, naphthylmethyl and the like.

The term “cycloalkyl,” unless otherwise specified, refers to cyclic alkyl groups of from 3 to 20 carbon atoms having a single cyclic ring or multiple condensed rings, which may optionally contain one or more olefinic bonds, unless otherwise constrained by the definition. Such cycloalkyl groups can include, for example, single ring structures, including cyclopropyl, cyclobutyl, cyclooctyl, cyclopentenyl, and the like or multiple ring structures, including adamantanyl, and bicyclo[2.2.1]heptane or cyclic alkyl groups to which is fused an aryl group, for example, indane, and the like, Spiro and fused ring structures can also be included. Cycloalkyl groups may be substituted further with one or more substituents selected from alkyl, alkenyl, alkynyl, alkoxy, cycloalkyl, cycloalkenyl, acyl, acylamino, acyloxy, alkoxycarbonylamino, azido, cyano, halogen, hydroxy, oxo, thiocarbonyl, carboxy, carboxyalkyl, arylthio, thiol, alkylthio, aryl, aralkyl, aryloxy, aminosulfonyl, aminocarbonylamino, —NR_{λ}R_{π}, —NHC(═O)NR_{λ}R_{π}, —NHC(═O)R_{λ}, —C(═O)NR_{λ}R_{π}, —O—C(═O)NR_{λ}R_{π}, nitro, heterocyclyl, heteroaryl, heterocyclylalkyl, heteroarylalkyl or SOₘR_{ψ} (wherein R_{λ}, R_{π}, m and R_{ψ} are the same as defined earlier). Unless otherwise constrained by the definition, cycloalkyl substituents optionally may be substituted further by 1-3 substituents selected from alkyl, alkenyl, alkynyl, carboxy, hydroxy, alkoxy, halogen, CF₃, —NR_{λ}R_{π}, —C(═O)NR_{λ}R_{π}, —NHC(═O)NR_{λ}R_{π}, —OC(═O)NR_{λ}R_{π}, cyano or —SOₘR_{ψ} (wherein R_{λ}, R_{π}, m and R_{ψ} are the same as defined earlier). “Cycloalkylalkyl” refers to alkyl-cycloalkyl group linked through alkyl portion, wherein the alkyl and cycloalkyl are the same as defined earlier.

The term “carboxy” as defined herein refers to —C(═O)OH.

The term “aryloxy” denotes the group O-aryl, wherein aryl is as defined above.

The term “heteroaryl,” unless otherwise specified, refers to an aromatic ring structure containing 5 or 6 ring atoms or a bicyclic or tricyclic aromatic group having from 8 to 10 ring atoms, with one or more heteroatom(s) independently selected from N, O or S optionally substituted with 1 to 4 substituent(s) selected from halogen (e.g., F, Cl, Br, I), hydroxy, alkyl, alkenyl, alkynyl, cycloalkyl, acyl, carboxy, aryl, alkoxy, aralkyl, cyano, nitro, heterocyclyl, heteroaryl, —NR_{λ}R_{π}, CH═NOH, —(CH₂)_{w}C(═O)R_{η} {wherein w is an integer from 0-4 and R_{η} is hydrogen, hydroxy, OR_{λ}, NR_{λ}R_{π}, —NHOR_{ω} or —NHOH}, —C(═O)NR_{λ}R_{π}, —NHC(═O)NR_{λ}R_{π}—, —SOₘR_{ψ}, —O—C(═O)NR_{λ}R_{π}, —O—C(═O)R_{λ}, or —O—C(═O)OR_{λ} (wherein m, R_{ψ}, R_{λ} and R_{π} are as defined earlier and R_{ω} is alkyl, cycloalkyl, aryl, heteroaryl, heterocyclyl, heteroarylalkyl or heterocyclylalkyl). Unless otherwise constrained by the definition, the substituents are attached to a ring atom, i.e., carbon or heteroatom in the ring. Examples of heteroaryl groups include oxazolyl, imidazolyl, pyrrolyl, 1,2,3-triazolyl, 1,2,4-triazolyl, tetrazolyl, thiazolyl, oxadiazolyl, benzoimidazolyl, thiadiazolyl, pyridinyl, pyridazinyl, pyrimidinyl, pyrazinyl, thienyl, isoxazolyl, triazinyl, furanyl, benzofuranyl, indolyl, benzthiazinyl, benzthiazinonyl, benzoxazinyl, benzoxazinonyl, quinazonyl, carbazolyl phenothiazinyl, phenoxazinyl, benzothiazolyl or benzoxazolyl, and the like.

The term “heterocyclyl,” unless otherwise specified, refers to a non-aromatic monocyclic or bicyclic cycloalkyl group having 5 to 10 atoms wherein 1 to 4 carbon atoms in a ring are replaced by heteroatoms selected from O, S or N, and optionally are benzofused or fused heteroaryl having 5-6 ring members and/or optionally are substituted, wherein the substituents are selected from halogen (e.g., F, Cl, Br, I), hydroxy, alkyl, alkenyl, alkynyl, cycloalkyl, acyl, optionally substituted aryl, alkoxy, alkaryl, cyano, nitro, oxo, carboxy, optionally substituted heterocyclyl, optionally substituted heterocyclylalkyl, optionally substituted heteroaryl, —O—C(═O)R_{λ}, —O—C(═O)OR_{λ}, —C(═O)NR_{λ}R_{π}, SOₘR_{ψ}, —O—C(═O)NR_{λ}R_{π}, —NHC(═O)NR_{λ}R_{π}, —NR_{λ}R_{π}, mercapto, haloalkyl, thioalkyl, —COOR_{ψ}, —COONHR_{λ}, —COR_{λ}, —NHSO₂R_{λ} or SO₂NHR_{λ} (wherein m, R_{ψ}, R_{λ} and R_{π} are as defined earlier) or guanidine. Heterocyclyl can optionally include rings having one or more double bonds. Such ring systems can be mono-, bi- or tricyclic. Carbonyl or sulfonyl group can replace carbon atom(s) of heterocyclyl. Unless otherwise constrained by the definition, the substituents are attached to the ring atom, i.e., carbon or heteroatom in the ring. Also, unless otherwise constrained by the definition, the heterocyclyl ring optionally may contain one or more olefinic bond(s). Examples of heterocyclyl groups include oxazolidinyl, tetrahydrofuranyl, dihydrofuranyl, benzoxazinyl, benzthiazinyl, imidazolyl, benzimidazolyl, tetrazolyl, carbaxolyl, indolyl, phenoxazinyl, phenothiazinyl, dihydropyridinyl, dihydroisoxazolyl, dihydrobenzofuryl, azabicyclohexyl, thiazolidinyl, dihydroindolyl, pyridinyl, isoindole 1,3-dione, piperidinyl, tetrahydropyranyl, piperazinyl, 3H-imidazo[4,5-b]pyridine, isoquinolinyl, 1H-pyrrolo[2,3-b]pyridine or piperazinyl and the like.

“Heteroarylalkyl” refers to heteroaryl (wherein heteroaryl is same as defined earlier) linked through alkyl (wherein alkyl is the same as defined above).

“Heterocyclylalkyl” refers to heterocyclyl (wherein heterocyclyl is same as defined earlier) linked through alkyl (wherein alkyl is the same as defined above).

“Acyl” refers to —C(═O)R″ wherein R″ is selected from the group hydrogen, alkyl, alkenyl, alkynyl, cycloalkyl, aryl, aralkyl, heteroaryl, heterocyclyl, heteroarylalkyl or heterocyclylalkyl.

“Thiocarbonyl” refers to —C(═S)H.

“Substituted thiocarbonyl” refers to —C(═S)R″ wherein R″ is selected is the same as defined earlier.

The term “leaving group” generally refers to groups that exhibit the desirable properties of being labile under the defined synthetic conditions and also, of being easily separated from synthetic products under defined conditions. Examples of such leaving groups includes but not limited to halogen (F, Cl, Br, I), triflates, tosylate, mesylates, alkoxy, thioalkoxy, hydroxy radicals and the like.

The term “protecting groups” refers to moieties that prevent chemical reaction at a location of a molecule intended to be left unaffected during chemical modification of such molecule. Unless otherwise specified, protecting groups may be used on groups, such as hydroxy, amino, or carboxy. Examples of protecting groups are found in T. W. Greene and P. G. M. Wuts, “Protective Groups in Organic Synthesis”, 2^{nd} Ed., John Wiley and Sons, New York, N.Y., which is incorporated herein by reference. The species of the carboxylic protecting groups, amino protecting groups or hydroxy protecting groups employed are not critical, as long as the derivatised moieties/moiety is/are stable to conditions of subsequent reactions and can be removed without disrupting the remainder of the molecule.

The term “pharmaceutically acceptable salts” refers to derivatives of compounds that can be modified by forming their corresponding acid or base salts. Examples of pharmaceutically acceptable salts include, but are not limited to, mineral or organic acids salts of basic residues (such as amines), or alkali or organic salts of acidic residues (such as carboxylic acids), and the like.

### DETAILED DESCRIPTION OF THE INVENTION

The compounds of the present invention may be prepared by techniques well known in the art and familiar to the person skilled in this field. In addition, the compounds of the present invention may be prepared, for example, following reaction sequence including that depicted below. The process described herein may be performed in appropriate alternate sequences to give the desired product.

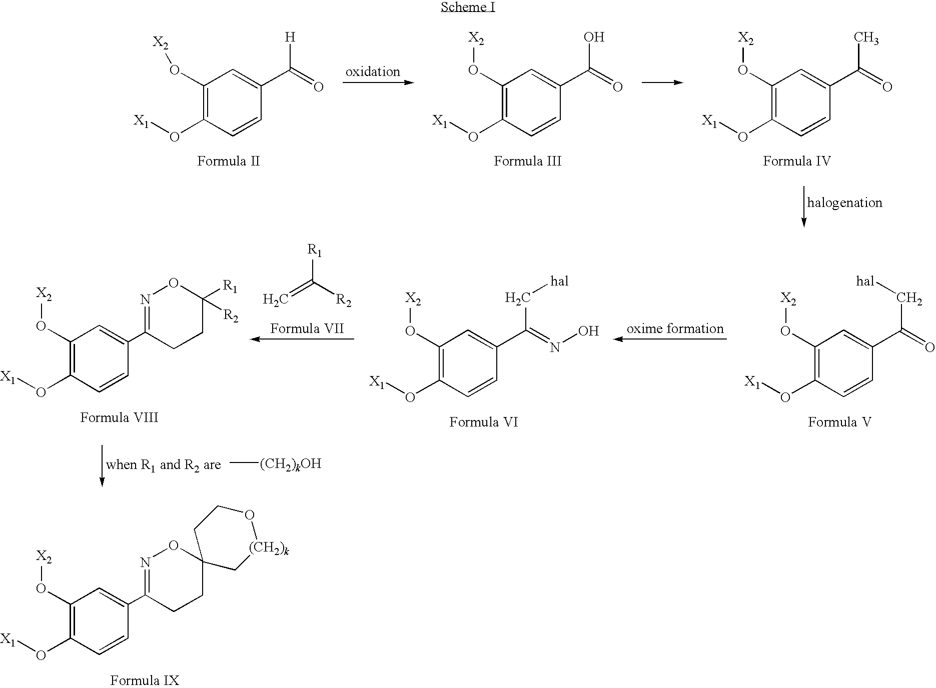

Compounds of Formula VIII can be prepared by methods, for example, shown in Scheme I. The compound of Formula II (wherein X₁ and X₂ are the same as defined earlier) can undergo oxidation to give a compound of Formula III, which can be converted to a compound of Formula IV, which can undergo halogenation to give a compound of Formula V (wherein hal is Br, Cl or I), which can be reacted with hydroxylamine hydrochloride to give a compound of Formula VI, which can be reacted with a compound of Formula VII (wherein R₁ and R₂ are the same as defined earlier) to give a compound of Formula VIII which can undergo cyclization (when R₁ and R₂ are —(CH₂)ₖOH wherein k is 1-4) to give a compound of Formula IX (wherein m is 0-2).

The oxidation of a compound of Formula II to give a compound of Formula III can be carried out with oxidizing agents such as, for example, sodium chlorite (NaClO₂), potassium chlorate (KClO₃), potassium perchlorate (KClO₄), potassium permanganate (KMnO₄), silver oxide (Ag₂O) or potassium dichromate in the presence of a solvent such as, for example, glacial acetic acid, acetone, water or acetic anhydride and in the presence of scavengers such as, for example, sulphamic acid, hydrazine, sodium sulphite or diethylhydroxyethanol.

The reaction of a compound of Formula III with methyl lithium to give a compound of Formula IV can be carried out in an organic solvent such as, for example, tetrahydrofuran, dimethylformamide, diethylether or dioxane in the presence of a catalyst such as, for example, trimethylchlorosilane, trimethylsilylimidazole, hexamethyldisilaze, bistrimethylsilylacetamide.

The halogenation of a compound of Formula IV to give a compound of Formula V can be carried out in the presence of halogenating agent such as, for example, benzyltrimethyl ammonium dichloroiodate, trimethyl chloro silane, sulfuryl chloride, trichloroisocyanuric acid, copper chloride, N-chlorosuccinimide, N-bromosuccinimide, or N-iodosuccinimide.

The compound of Formula V can be reacted with hydroxylamine hydrochloride in the presence of an acetate, such as sodium acetate, to yield the compound of Formula VI.

The compound of Formula VI can be reacted with a compound of Formula VII to give a compound of Formula VIII in an organic solvent such as tetrahydrofuran, dichloromethane, acetonitrile, diethylether, nitomethane, dimethylformamide, chloroform, or carbon tetrachloride, in the presence of a base such as sodium carbonate, potassium carbonate, sodium acetate, or sodium hydrogen carbonate.

The compound of Formula VIII can undergo ring cyclization to give a compound of Formula IX in an organic solvent, for example, tetrahydrofuran, dimethylformamide, dioxane or diethyl ether, with reagents, for example, diisopropyldiazadicarboxylate (DIAD), or diethyldiazadicarboxylate (DEAD), in the presence of catalyst, for example triphenyl phosphine, tri-tertbutyl phosphine, or tricyclohexyl phosphine.

Particular illustrative compounds which can be prepared, for example, following Scheme I include:
3-(3-Cyclopentyloxy-4-methoxy-phenyl)-1-oxa-2-aza-spiro[5.5]undec-2-ene (Compound No. 1),
8-(3-Cyclopentyloxy-4-methoxy-phenyl)-6-oxa-7-aza-spiro[4.5]dec-7-ene (Compound No. 2),
7-(3-Cyclopentyloxy-4-methoxy-phenyl)-5-oxa-6-aza-spiro[3.5]non-6-ene (Compound No. 3),
[3-(3-Cyclopentyloxy-4-methoxy-phenyl)-6-hydroxymethyl-5,6-dihydro-4H-[1,2]oxazin-6-yl]methanol (Compound No. 4),
3-(3-Cyclopentyloxy-4-methoxy-phenyl)-6-(2-oxo-propyl)-5,6-dihydro-4H-[1,2]oxazin-6-carboxylic acid methyl ester (Compound No. 5),
3-(3-Cyclopentyloxy-4-methoxy-phenyl)-5,6-dihydro-4H-[1,2]oxazin-6-carboxylic acid ethyl ester (Compound No. 6),
3-(3-Cyclopentyloxy-4-methoxy-phenyl)-5,6-dihydro-4H-[1,2]oxazin-6-carboxylic acid (Compound No. 7),
2-[3-(3-Cyclopentyloxy-4-methoxy-phenyl)-5,6-dihydro-4H-[1,2]oxazin-6-yl]-ethanol (Compound No. 8),
8-(3-Cyclopentyloxy-4-methoxy-phenyl)-2,6-dioxa-7-aza-spiro[4,5]dec-7-ene (Compound No. 9), or
   their pharmaceutically acceptable salts, pharmaceutically acceptable solvates, enantiomers, diastereomers or N-oxides.

In the above schemes, where specific bases, condensing agents, hydrolyzing agents, solvents, etc. known to those skilled in the art may be used. Similarly, the reaction temperature and duration of the reaction may be adjusted according to the desired needs.

Because of their valuable pharmacological properties, the compounds described herein may be administered to an animal for treatment orally, or by a parenteral route. The pharmaceutical compositions described herein can be produced and administered in dosage units, each unit containing a certain amount of at least one compound described herein and/or at least one physiologically acceptable addition salt thereof. The dosage may be varied over extremely wide limits, as the compounds are effective at low dosage levels and relatively free of toxicity. The compounds may be administered in the low micromolar concentration, which is therapeutically effective, and the dosage may be increased as desired up to the maximum dosage tolerated by the patient.

The compounds described herein can be produced and formulated as their enantiomers, diastereomers, N-oxides, polymorphs, solvates and pharmaceutically acceptable salts, as well as metabolites having the same type of activity. Pharmaceutical compositions comprising the molecules of Formula I or metabolites, enantiomers, diastereomers, N-oxides, polymorphs, solvates or pharmaceutically acceptable salts thereof, in combination with pharmaceutically acceptable carrier and optionally included excipient can also be produced.

Where desired, the compounds of Formula I and or their pharmaceutically acceptable salts, pharmaceutically acceptable solvates, stereoisomers, tautomers, racemates, prodrugs, metabolites, polymorphs or N-oxides may be advantageously used in combination with one or more other therapeutic agents. Examples of other therapeutic agents, which may be used in combination with compounds of Formula I of this invention and/or their pharmaceutically acceptable salts, pharmaceutically acceptable solvates, stereoisomers, tautomers, racemates, prodrugs, metabolites, polymorphs or N-oxides include corticosteroids, beta agonists, leukotriene antagonists, 5-lipoxygenase inhibitors, chemokine inhibitors and muscarinic receptor antagonists.

Examples set forth below demonstrate general synthetic procedures for the preparation of representative compounds. The examples are provided to illustrate particular aspects of the disclosure and do not limit the scope of the claims.

### EXAMPLES

### General Synthesis for the Preparation of Compound of Formula VI

### Step a: Synthesis of Compound of Formula III

A solution of sodium chlorite (8.7 gm, 0.079 moles) was taken in water (19.5 ml) followed by the addition of compound of Formula II (prepared following the procedure as described in J. Med. Chem., (1994), 37, 1696-1703) (13 gm, 0.059 moles), sulphamic acid (7.7 gm, 0.0796 moles) and glacial acetic acid (50 ml), under cooling at 0° C. After completion of addition, reaction mixture was stirred for one hour at room temperature and then diluted with water. The precipitated solid product was filtered, washed with water and hexane and dried under vacuum to furnish the title compound.

### Step b: Synthesis of Compound of Formula IV

The solution of the compound obtained from step a above (9 gm, 0.038 moles) in dry tetrahydrofuran (100 ml) was cooled to 0° C. followed by the addition of methyl lithium (1.66 gm, 0.076 m) slowly at 0° C. The solution was then stirred at 0° C. for 2 hour. Trimethylchlorosilane (8.25 gm, 0.076 m) was added dropwise into the solution. After addition, reaction mixture was stirred at room temperature for another 40 minutes. Reaction mixture was quenched with ammonium chloride solution. The solvent was evaporated under reduced pressure and the residue thus obtained diluted by addition of water. The mixture was extracted with ethyl acetate. The organic layer evaporated to under reduced pressure and the residue thus obtained was purified by column chromatography to furnish the title compound.

### Step c: Synthesis of Compound of Formula V

Benzyltrimethyl ammonium dichloroiodate (800 mg, 2.02 mM) was added to a solution of the compound obtained from step b above (250 mg, 1.068 mM) in dichloromethane (25 ml) and methanol (10 ml). The reaction mixture was refluxed for 3 hours. Solvent was evaporated under reduced pressure and aqueous solution of sodium bicarbonate (5%, 30 ml) was added to the residue thus obtained. The mixture was extracted with ethyl acetate and the organic layer was dried over anhydrous sodium sulphate, filtered and evaporated under reduced pressure to furnish the title compound.

### Step d: Synthesis of Compound of Formula VI

To a solution of hydroxylamine hydrochloride (194.58 mg, 2.82 mM) in methanol (20 ml) was added sodium acetate (231.24 mg, 2.82 mM) followed by the addition of a compound obtained from step c (252 mg, 1.069 mM) above in methanol (5 ml) dropwise to it. The reaction mixture was stirred for 1 hour. To the resulting reaction mixture was added water and the organic solvent was evaporated under reduced pressure. The mixture was extracted with ethyl acetate. Organic layer was concentrated under reduced pressure and the residue thus obtained was purified by column chromatography to furnish the title compound.

The following compound was prepared similarly,

### 2-Chloro-1-(3-Cyclopentyloxy-4-methoxy-phenyl)ethanone oxime

¹HNMR: δ 8.21 (s, 1H), 7.28 (d, 1H), 7.22 (m, 1H), 6.8 (d, 1H), 4.82 (m, 1H), 4.65 (s, 2H), 3.9 (s, 3H), 1.99-4.60 (m, 8H).

### Example 1

### Synthesis 3-(3-Cyclopentyloxy-4-methoxy-phenyl)-5,6-dihydro-4H-[1,2]oxazin-6-carboxylic acid ethyl ester (Compound No. 6)

To a solution of the compound 2-chloro-1-(3-Cyclopentyloxy-4-methoxy-phenyl)-ethanone oxime (100 mg, 0.353 mM) and ethyl acrylate (212 mg (2.12 m mol) in dry tetrahydrofuran (3 ml) was added sodium carbonate (230 mg, 2.173 mM) and stirred the reaction mixture for 40 hours. Tetrahydrofuran was evaporated under reduced pressure and the compound was extracted with dichloromethane. Organic layer was concentrated under reduced pressure to obtain the crude mixture, which was then purified to furnish the title compound. Yield: 35 mg. Mass (m/z): 348 (M⁺+1). ¹H NMR: δ 7.4 (d, 1H), 7.0 (m, 1H), 6.82 (d, 1H), 4.8 (m, 1H), 4.5 (m, 1H), 4.3 (m, 2H), 3.86 (s, 3H), 2.6 (t, 2H), 2.3 (m, 2H), 1.9-1.6 (m, 8H) 1.3 (m, 3H).

The following compounds were prepared similarly,
3-(3-Cyclopentyloxy-4-methoxy-phenyl)-1-oxa-2-aza-spiro[5.5]undec-2-ene (Compound No. 1) Mass (m/z): 344 (M⁺+1),
8-(3-Cyclopentyloxy-4-methoxy-phenyl)-6-oxa-7-aza-spiro[4.5]dec-7-ene (Compound No. 2) Mass (m/z): 330 (M⁺+1),
7-(3-Cyclopentyloxy-4-methoxyphenyl)-5-oxa-6-aza-spiro[3.5]non-6-ene (Compound No. 3) Mass (m/z): 316 (M⁺+1),
[3-(3-Cyclopentyloxy-4-methoxy-phenyl)-6-hydroxymethyl-5,6-dihydro-4H-[1,2]oxazin-6-yl]methanol (Compound No. 4); Mass (m/z): 336 (M⁺+1); and
3-(3-Cyclopentyloxy-4-methoxy-phenyl)-6-(2-oxo-propyl)-5,6-dihydro-4H-[1,2]oxazin-6-carboxylic acid methyl ester (Compound No. 5) Mass (m/z): 406 (M⁺+1).

### Example 2

### Synthesis of 3-(3-Cyclopentyloxy-4-methoxy-phenyl)-5,6-dihydro-4H-[1,2]oxazin-6-carboxylic acid (Compound No. 7)

To a solution of Compound No. 6 (65 mg, 0.1873 mM) in tetrahydrofuran was added aqueous lithium hydroxide (in 6 ml H₂O, 15.7 mg, 0.3746 mM) solution and the reaction mixture was stirred at 60° C. for 3 hours. Tetrahydrofuran was evaporated under reduced pressure and the residue thus obtained was diluted with water followed by washing with ethyl acetate to remove organic impurities. The aqueous layer was then neutralized with hydrochloric acid (1N) to attain the pH of aqueous solution to 4. The mixture was extracted with ethyl acetate to furnish the title compound. Yield: 59 mg.

Mass (m/z): 320 (M⁺+1). ¹H NMR: δ 7.3 (d, 1H), 7.12 (m, 1H), 6.83 (d, 1H), 4.8 (m, 1H), 4.65 (m, 1H), 3.87 (s, 3H), 2.66 (t, 2H), 2.36 (m, 2H), 1.9-1.6 (m, 8H).

### Example 3

### Synthesis of 2-[3-(3-Cyclopentyloxy-4-methoxy-phenyl)-5,6-dihydro-4H-[1,2]oxazin-6-yl]-ethanol (Compound No. 8)

To a solution of Compound No. 5 (70 mg, 0.1728 mM) in tetrahydrofuran (18 ml) was added sodium borohydride (26.27 mg, 0.6913 mM) and reaction mixture was stirred for 30 minutes. To the resulting reaction mixture was added methanol (2-4 drops). Reaction mixture was then stirred for another 3 hours. The reaction mixture was quenched with hydrochloric acid (1N) till solution attained pH 7. Tetrahydrofuran was evaporated under reduced pressure followed by water. The mixture was extracted with ethyl acetate and the organic layer was concentrated under reduced pressure. The residue thus obtained was purified by column chromatography to furnish the title compound. Yield: 36 mg. ¹H NMR: δ 7.39 (d, 1H), 7.13 (m, 1H), 6.83 (d, 1H), 4.8 (m, 1H), 3.9 (m, 1H), 3.8 (s, 3H), 3.7 (m, 1H), 3.6 (m, 2H), 2.5 (m, 2H), 2.0 (m, 2H), 1.9-1.6 (m, 10H).

### Example 4

### Synthesis of 8-(3-Cyclopentyloxy-4-methoxy-phenyl)-2,6-dioxa-7-aza-spiro[4,5]dec-7-ene (Compound No. 9)

To a solution of the Compound No. 8 (90 mg, 0.259 mM) in tetrahydrofuran was added triphenylphosphine (81.07 mg, 0.309 mM) and succinimide (30.61 mg, 0.309 mM) followed by the addition of diisopropyldiazadicarboxylate (57.10 mg, 0.2827 mM). The reaction mixture was stirred at room temperature for overnight. The organic solvent was removed under reduced pressure and the residue thus obtained was purified by column chromatography to furnish the title compound. Yield: 38 mg, Mass (m/z): 332 (M⁺+1). ¹H NMR: δ 7.4 (d, 1H), 7.13 (m, 1H), 6.83 (d, 1H), 4.8 (m, 1H), 3.86 (s, 3H), 3.8 (s, 3H), 3.7 (s, 1H), 2.6 (m, 2H), 2.09 (m, 1H), 2.04 (m, 2H), 1.9-1.6 (m, 9H).

### Example 5

### Biological Assay

### PDE-IV Enzyme Assay

The efficacy of compounds of PDE-4 inhibitors was determined by an enzyme assay using cell lysate of HEK293 cells transfected with PDE4B2 plasmids as the PDE4B source. The enzyme reaction was carried out in the presence of cAMP (1 μM) at 30° C. in the presence or absence of test compound for 45-60 minutes. An aliquot of this reaction mixture was taken further for the ELISA assay and the protocol of the kit followed to determine level of cAMP in the sample. The concentration of the cAMP in the sample directly correlates with the degree of PDE-4 enzyme inhibition. Results were expressed as percent control and the IC₅₀ values of test compounds were reported.

IC₅₀ values of the specifically disclosed compounds were found to be in the range of lower μM to nM concentration, for example, from about 5 nM to about 3.7 μM, or for example, from about 5 nM to about 500 nM, or from about 5 nM to about 200 nM, or from about 5 nM to about 30 nM.

### Cell Based Assay for TNF-α Release

### Method of Isolation of Human Peripheral Blood Mononuclear Cells:

Human whole blood was collected in vacutainer tubes containing heparin or EDTA as an anti coagulant. The blood was diluted (1:1) in sterile phosphate buffered saline and 10 ml was carefully layered over 5 ml Ficoll Hypaque gradient (density 1.077 g/ml) in a 15 ml conical centrifuge tube. The sample was centrifuged at 3000 rpm for 25 minutes in a swing-out rotor at room temperature. After centrifugation, interface of cells were collected, diluted at least 1:5 with PBS and washed three times by centrifugation at 2500 rpm for 10 minutes at room temperature. The cells were resuspended in serum free RPMI 1640 medium at a concentration of 2 million cells/ml.

### LPS Stimulation of Human PBMNC's:

PBMN cells (0.1 ml; 2 million/ml) were co-incubated with 20 ml of compound (final DMSO concentration of 0.2%) for 10 minutes in a flat bottom 96-well microliter plate. Compounds were dissolved in DMSO initially and diluted in medium for a final concentration of 0.2% DMSO. LPS (1 mg/ml, final concentration) was then added at a volume of 10 μl per well. After 30 minutes, 20 μl of fetal calf serum (final concentration of 10%) was added to each well. Cultures were incubated overnight at 3° C. in an atmosphere of 5% CO₂ and 95% air. Supernatant were then removed and tested by ELISA for TNF-α release using a commercial kit (e.g. BD Biosciences). The level of TNF-α in treated wells was compared with the vehicle treated controls and inhibitory potency of compound was expressed as IC₅₀ values calculated by using Graph pad prism.

$$Percent inhibition = 100 - \frac{Percent TNF \text{-} α drug treated}{Percent TNF \text{-} α in vehicle treated} \times 100$$

IC₅₀ values of the specifically disclosed compounds were found to be in the range of lower μM to nM concentration, for example, from about 10 μM to about 3.9 μM.

We claim:

1. A compound having the structure of Formula I: 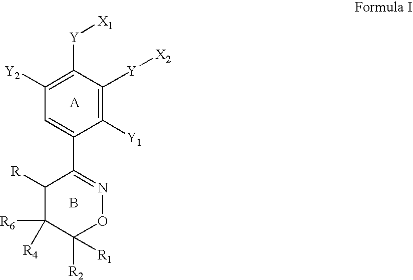
and its pharmaceutically acceptable salts, pharmaceutically acceptable solvates, enantiomers, diastereomers or N-oxides, wherein
R₁ is hydrogen, alkyl, heterocyclyl, —(CH₂)₁₋₄OR′; —C(═O)NRₓR_{y} or —(CH₂)ₘ—C(═O)R₃;
m is an integer from 0-4;
R₂ is —(CH₂)ₘC(═O)R₃, —(CH₂)₁₋₄OR′, —C(═O)NRₓR_{y} or R₁ and R₂ together forms an optionally substituted cycloalkyl or heterocyclyl ring system;
R₃ is OR′ or R′;
R₄ is hydrogen, alkyl, —OR₅, halogen, —NH₂, substituted amino, cyano, carboxy, or —C(═O)NRₓR_{y}, or R₂ and R₄ together join to form an optionally substituted cycloalkyl or heterocyclyl ring system;
R₆ is hydrogen, alkyl, alkenyl, alkynyl, —OR₅, halogen, cyano, —NH₂ or substituted amino, or R₄ and R₆ together join to form an optionally substituted cycloalkyl or heterocyclyl ring system;
R′ is alkyl, alkenyl, alkynyl, aryl, cycloalkyl, heteroaryl, heterocyclyl, heteroarylalkyl, heterocyclylalkyl or aralkyl;
R is R′, —OR₅, halogen, cyano, —NH₂ or substituted amino;
X₁ and X₂ are each independently hydrogen, alkyl, alkaryl, cycloalkyl, heterocyclyl, heteroaryl, heterocyclylalkyl or heteroarylalkyl;
Y is an oxygen atom, a sulphur atom, or —NR;
Y₁ and Y₂ are each independently hydrogen, alkyl, —OR, —SR, or —NHR,
wherein any of Y₁ and X₂ & X₁ and Y₂ together optionally form a cyclic ring fused with the ring A, the ring containing 3-5 carbon atoms within the ring and having 1-3 heteroatoms;
X₁ and X₂ can together optionally form a ring fused with ring A, the ring containing 3-5 carbon atoms within the ring and having 2-3 heteroatoms; and
Rₓ and R_{y} are independently hydrogen, alkyl, alkenyl of three to six carbon atoms, alkynyl of three to six carbon atoms, cycloalkyl, —SO₂R₅ (wherein R₅ is hydrogen, alkyl, alkenyl, alkynyl, aryl, cycloalkyl, alkaryl, heteroaryl, heteroarylalkyl, heterocyclyl or heterocyclylalkyl), aryl, alkaryl, heteroaryl, heterocyclyl, heteroarylalkyl, or heterocyclylalkyl.

2. A compound which is selected from:
3-(3-Cyclopentyloxy-4-methoxy-phenyl)-1-oxa-2-aza-spiro[5.5]undec-2-ene (Compound No. 1),
8-(3-Cyclopentyloxy-4-methoxy-phenyl)-6-oxa-7-aza-spiro[4.5]dec-7-ene (Compound No. 2),
7-(3-Cyclopentyloxy-4-methoxy-phenyl)-5-oxa-6-aza-spiro[3.5]non-6-ene (Compound No. 3),
[3-(3-Cyclopentyloxy-4-methoxy-phenyl)-6-hydroxymethyl-5,6-dihydro-4H-[1,2]oxazin-6-yl]methanol (Compound No. 4),
3-(3-Cyclopentyloxy-4-methoxy-phenyl)-6-(2-oxo-propyl)-5,6-dihydro-4H-[1,2]oxazin-6-carboxylic acid methyl ester (Compound No. 5),
3-(3-Cyclopentyloxy-4-methoxy-phenyl)-5,6-dihydro-4H-[1,2]oxazin-6-carboxylic acid ethyl ester (Compound No. 6),
3-(3-Cyclopentyloxy-4-methoxyphenyl)-5,6-dihydro-4H-[1,2]oxazin-6-carboxylic acid (Compound No. 7),
2-[3-(3-Cyclopentyloxy-4-methoxy-phenyl)-5,6-dihydro-4H-[1,2]oxazin-6-yl]-ethanol (Compound No. 8),
8-(3-Cyclopentyloxy-4-methoxy-phenyl)-2,6-dioxa-7-aza-spiro[4,5]dec-7-ene (Compound No. 9), and
their pharmaceutically acceptable salts, pharmaceutically acceptable solvates, enantiomers, diastereomers or N-oxides.

3. A pharmaceutical composition comprising a therapeutically effective amount of a compound as defined in claim 1 together with pharmaceutically acceptable carriers, excipients or diluents.

4. The use of compounds according to claim 1 for the manufacture of medicament for treating or preventing CNS disorders, inflammatory diseases selected from AIDS, asthma, arthritis, bronchitis, chronic obstructive pulmonary disease (COPD), psoriasis, allergic rhinitis, shock, atopic dermatitis, Crohn's disease, adult respiratory distress syndrome (ARDS), eosinophilic granuloma, allergic conjunctivitis, osteoarthritis, ulcerative colitis and other inflammatory diseases especially in humans.

5. The use of the compounds as described in claim 4 for the manufacture of medicament for treating or preventing inflammatory condition in an animal or human.

6. The use of compounds according to claim 4 and 5 wherein the disease or disorder is mediated through phosphodiesterase type IV.

7. A method for preparing a compound of Formula VII and its pharmaceutically acceptable salts, pharmaceutically acceptable solvates, enantiomers, diastereomers or N-oxides wherein the method comprises:
a. oxidizing a compound of Formula II 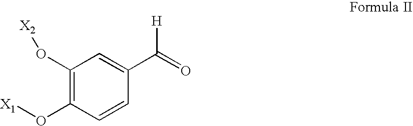
to give a compound of Formula III; 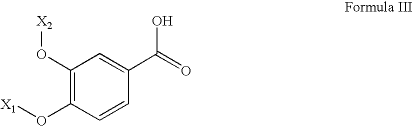
b. converting the compound of Formula III to give a compound of Formula IV; 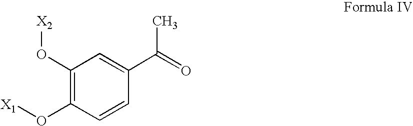
c. halogenating the compound of Formula IV to give a compound of Formula V; 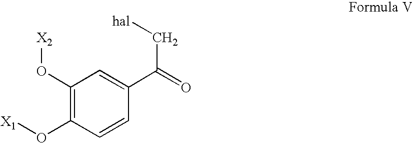
d. reacting the compound of Formula V with hydroxyl amine hydrochloride to give a compound of Formula VI; 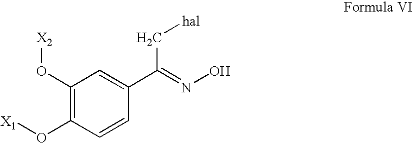
e. reacting the compound of Formula VI with a compound of Formula VII 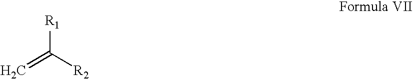
to give a compound of Formula VIII; and 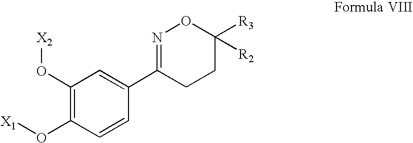
f. cyclizing the compound of Formula VIII to give a compound of Formula IX, 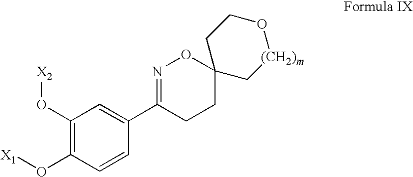
wherein
R₁ is hydrogen, alkyl, heterocyclyl, —(CH₂)₁₋₄OR′, —C(═O)NRₓR_{y} or —(CH₂)ₘ—C(═O)R₃;
R₂ is —(CH₂)ₘC(═O)R₃, —(CH₂)₁₋₄OR′, —C(═O)NRₓR_{y} or R₁ and R₂ together form an optionally substituted cycloalkyl or heterocyclyl ring system;
R₃ is OR′ or R′;
R′ is alkyl, alkenyl, alkynyl, aryl, cycloalkyl, heteroaryl, heterocyclyl, heteroarylalkyl, heterocyclylalkyl or aralkyl;
X₁ and X₂ each independently are hydrogen, alkyl, alkaryl, cycloalkyl, heterocyclyl, heteroaryl, heterocyclylalkyl or heteroarylalkyl;
X₁ and X₂ can together optionally form a ring fused with ring A, the ring containing 3-5 carbon atoms within the ring and having 2-3 heteroatoms;
Rₓ and R_{y} are independently hydrogen, alkyl, alkenyl of three to six carbon atoms, alkynyl of three to six carbon atoms, cycloalkyl, —SO₂R₅ (wherein R₅ is hydrogen, alkyl, alkenyl, alkynyl, aryl, cycloalkyl, alkaryl, heteroaryl, heteroarylalkyl, heterocyclyl or heterocyclylalkyl), aryl, alkaryl, heteroaryl, heterocyclyl, heteroarylalkyl, and heterocyclylalkyl.
